(19) United States
(12) Patent Application Publication
(10) Pub. No.: US 2009/0117050 A1
(43) Pub. Date: May 7, 2009

(54) STEM CELL TARGETING OF CANCER, METHODS AND COMPOSITIONS THEREFOR

(75) Inventors: Craig Cady, Springfield, IL (US); Mary McAsey, Springfield, IL (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: Bradley University, Peora, IL (US)

(21) Appl. No.: 12/253,685

(22) Filed: Oct. 17, 2008

Related U.S. Application Data
(60) Provisional application No. 60/980,764, filed on Oct. 17, 2007.

(51) Int. Cl. A61B 5/055; A61K 35/12; A61P 35/00

(52) U.S. Cl. 424/9.3; 424/93.7; 424/93.21

(57) ABSTRACT

Disclosed are methods of detecting and treating a cancer such as an ovarian cancer using stem cells. Detection methods include administering a plurality of labeled stem cells to a subject having, or suspected of having, a cancer; and detecting the distribution of the stem cells. In some configurations, the label can be a nanoparticle such as a mono-crystalline iron oxide, which can be detected by magnetic resonance imaging. Treatment methods include administering a plurality of stem cells comprising a therapeutic agent such as an enzyme which activates a prodrug. In some configurations, the stem cells harbor a nucleic acid sequence encoding a cytosine deaminase, the cells express the enzyme, and the treatment further includes administering the prodrug 5-fluorocytosine, which is converted by the cytosine deaminase to the cytotoxic metabolite, 5-fluorouracil (5-FU).

### CROSS-REFERENCE TO RELATED APPLICATIONS

This application claims priority to U.S. Provisional Patent Application Ser. No. 60/980,764, filed Oct. 17, 2007, the entire disclosure of which is incorporated herein by reference.

### INCORPORATION-BY-REFERENCE OF SEQUENCE LISTING IN COMPUTER READABLE FORM

The Sequence Listing, which is a part of the present disclosure, includes a computer readable form file entitled “SNR60000213_{—}0011_Sequence_listing.txt” comprising disclosed nucleotide and/or amino acid sequences submitted via EFS-Web. The subject matter of the Sequence Listing is incorporated herein by reference in its entirety.

### INTRODUCTION

Cancer remains a significant health problem. Ovarian cancer, in particular, is a leading cause of gynecologic cancer death in the western world. It is often diagnosed at an advanced stage (III-IV). 60% of diagnosed cases are fatal, and 80-90% originate from the ovarian surface epithelium.

Current treatments for ovarian cancer include total abdominal hysterectomy/bilateral salpingo-oophorectomy (TAH/BSO), debulking and staging laparotomy, systemic or intraperitoneal chemotherapy, and/or radiation therapy. However, various problems are associated with these treatments, such as toxic effects on normal tissues, patient fatigue/malaise, and long-term morbidity. The 5-year survival rate for advanced stage ovarian cancer has not changed over the past 30 years.

In the treatment of cancers, it is recognized that tumor hypoxia can be a major obstacle to effective treatment. Tumor hypoxia is known to up-regulate proto-oncogenes, and promote selection of the most clinically aggressive phenotypes. Furthermore, hypoxic tumors can be difficult to treat due to impeded drug delivery. (Harris, A. L., Nat'l. Rev. Cancer 2: 38-47, 2002; Axelson, H., Semin Cell Dev Biol. 16: 554-63, 2005).

Stem cells are used in the treatment of diseases such as lupus, sickle cell anemia, leukemia and some other diseases. In particular, autologous stem cells are readily obtained and are non-immunogenic to the donor/recipient. In addition, stem cells are known to migrate to areas of hypoxic stress or injury in some biological systems. For example, bone marrow stem cells migrate to cardiac infarcts (Pittenger, Circ. Res. 95: 9, 2004), and neural stem cells migrate to gliomas (Aboody, Neuro-oncol. 8: 119, 2006; Aboody, Proc. Nat'l Acad. Sci. USA 97: 12846-12851, 2000). Furthermore, human bone marrow mesenchymal stem cells (hMSCs) are recruited to stressed or injured tissues. (Annabi, Stem Cells 21: 337, 2003; Studeny, M., J. Nat'l. Cancer Inst. 96: 1593-1603, 2004; Khakoo, A. Y. JEM. 203: 1235-1247, 2006.). U.S. Pat. No. 7,015,037 to Furcht et al. describes using multipotent adult stem cells (MASCs) for treating cancer in a subject, in which the MASCs are genetically altered to express a tumoricidal protein, an anti-angiogenic protein, or a protein expressed on the surface of a tumor cell in conjunction with a protein associated with stimulation of an immune response to antigen. US Patent Application Publications 2005/0019313 and 2005/0115213 of Snyder et al. disclose neural stem cells transfected with DNA encoding cytosine deaminase and using them to treat primary brain tumors, neuroblastoma, melanoma and prostate cancers in model systems. However, stem cells have not been used in the treatment of many cancers such as ovarian cancer.

### SUMMARY

The present inventors disclose herein methods of detecting cancers such as ovarian cancers. Such cancers can be hypoxic tumors. In various aspects, these methods comprise administering a plurality of labeled stem cells to a subject having, or suspected of having, a cancer, and detecting the distribution of the stem cells. In various configurations, the stem cells can comprise a label which can be detected using methods and equipment well known to skilled artisans. Such labels and methods include, without limitation, a fluorophore label such as a fluorescent protein such as a GFP, fluorescein, or rhodamine which can be detected by optical methods; detection of a radioisotope label such as ¹⁹F using positron emission tomography (PET), or detection of nanoparticles, such as mono-crystalline iron oxide nanoparticles (MIONs) (Frank, J. A., Radiology 228: 480-487, 2003), using magnetic resonance imaging (MRI). In various aspects, stem cells which can be used for detecting a cancer can be bone marrow stem cells (BMSCs). Hence, in various aspects, a tumor, if present, can be imaged, and a diagnosis can be made, by imaging the distribution of labeled stem cells within a subject following administration of the stem cells to the subject.

In addition, aspects of the present teaching include bone marrow stem cells comprising mono-crystalline iron oxide nanoparticles (MIONs). In some configurations, the stem cells can be autologous to an intended recipient.

In various configurations, a cancer which can be detected using compositions and methods of the present teachings can be an ovarian cancer. In various configurations, the BMSCs can be autologous or heterologous to a subject, and the cancer can be an ovarian cancer. Furthermore, a subject can be any mammal, such as but not limited to, a human in need of diagnosis or therapy for a cancer; a companion animal such as a cat or dog; an agricultural animal such as a cow, a sheep or a pig, or a laboratory animal such as a mouse or rat.

In other aspects, the present inventors disclose methods and compositions for treating a cancer, such as an ovarian cancer. These methods comprise administering to a subject in need of treatment a therapeutically effective amount of stem cells comprising a therapeutic agent. In various configurations, a therapeutic agent comprised by stem cells can be an enzyme which activates a prodrug, such as, without limitation, the enzyme cytosine deaminase, which can activate the prodrug 5-fluorocytosine (5-FC) to the cytotoxic metabolite, 5-fluorouracil (5-FU). (See, e.g., Miller, C. R., Cancer Research 62, 773-780, 2002). In some configurations, the stem cells can comprise a nucleic acid encoding the therapeutic agent. Without limitation, stem cells of these aspects can comprise a vector comprising a nucleic acid, wherein the nucleic acid comprises a promoter operably linked to a nucleic acid sequence encoding an therapeutic agent such as the enzyme cytosine deaminase. The vector can be any vector known to skilled artisans, such as, without limitation, a virus, a plasmid, or a fragment thereof. Without limitation, a vector can be a plasmid comprising a retroviral pBabePuro backbone (Morgenstern, J. P., Nucl. Acids Res. 18: 3587-3596, 1990; Aboody, K. S., Proc. Nat'l Acad. Sci. USA 97: 12846-12851, 2000).

In various configurations, stem cells comprising such vectors express the encoded cytosine deaminase.

In various configurations of the disclosed treatment methods, the stem cells can be bone marrow stem cells (BMSCs), which can be autologous or heterologous to the subject. Furthermore, in various configurations, subject can be any mammal, such as but not limited to, a human in need of diagnosis or therapy for a cancer; a companion animal such as a cat or dog; an agricultural animal such as a cow, a sheep or a pig, or a laboratory animal such as a mouse or rat.

In various aspects, a treatment of the present teachings can further comprise administering a therapeutically effective amount of a prodrug to a subject. In addition, some aspects of disclosed methods for treating a cancer such as an ovarian cancer can include obtaining the BMSCs from the subject, and infecting, transfecting, or transforming or the BMSCs with a vector comprising a polynucleotide comprising a promoter operably linked to a sequence encoding an enzyme which activates a prodrug, such as, without limitation, an E. coli cytosine deaminase (Danielsen, S., Mol. Microbiol. 6: 1335-1344, 1992).

### BRIEF DESCRIPTION OF THE DRAWINGS

FIG. 1. HBMSC migrated through the 8 μm pores on the Matrigel-coated membrane. A) Fewer HBMSC migrated towards media without FBS. B) Significantly more HBMSC migrated towards media containing SKOV3 ovarian carcinoma cells. 20× magnification.

FIG. 2. Bone marrow stem cells migrated in response to conditioned media from ovarian cancer cells. A) Migration of HBMSC significantly (p<0.01) increased in the presence of SKOV3 ovarian carcinoma cells and SKOV3 cell conditioned media (CM)>40% compared to migration towards media without FBS (No serum) ANOVA followed by Dunnett's multiple comparison test. B) UCBSC show higher migration towards SKOV3 cells in contrast to migration toward media without FBS (No FBS). Bars represent means±SEM.

FIG. 3. Human bone marrow stem cells migrated to Hey spheroids following 24 hours of co-culture. A) Multicellular tumor spheroids of HEY ovarian cancer cells. B) CFDA label HBMSC migrating toward, surrounding, and interacting with the tumor spheroid (negative image of original).

FIG. 4. Co-cultures of HBMSC and SKOV3 cells. A) CFDA labeled HBMSC (FITC). B) Nuclei of both HBMSC and SKOV3 cells labeled with bisbenzamide (DAPI) (negative image of original).

FIG. 5. Human bone marrow stem cells exhibit greater migration when co-cultured with HEY cells. HBMSC migration is significantly greater after two days in vitro (DIV) co-culture with SKOV3 ovarian cancer cells. (p<0.001, ANOVA followed by Bonferroni's multiple comparison tests.)

FIG. 6 presents the results of some chemotaxis migration assays.

FIG. 7 presents the results of some Matrigel invasion assays.

### DETAILED DESCRIPTION

The present inventors disclose herein methods of detecting cancers such as ovarian cancers. The cancers can comprise hypoxic tumors. Methods of the present teachings involve administering a plurality of labeled stem cells to a subject having, or suspected of having, a cancer, and detecting the distribution of the stem cells. The labeled stem cells can serve as probes for detecting a tumor. The administration can be by standard methods well known to skilled artisans, such as, without limitation by intravenous or intraarterial injection. In various configurations, the stem cells can comprise a label which can be detected using methods and equipment well known to skilled artisans. Such labels and methods include, without limitation, a fluorophore label such as a fluorescent protein such as a GFP, fluorescein, or rhodamine which can be detected by optical methods; detection of a radioisotope label such as ¹⁹F using positron emission tomography (PET), or detection of nanoparticles, such as mono-crystalline iron oxide nanoparticles (MIONs), using magnetic resonance imaging (MRI) (e.g., Song, M., Korean J. Radiol. 8: 365-371, 2007). In various aspects, stem cells which can be used for detecting a cancer can be bone marrow stem cells (BMSCs). Hence, in various aspects, a tumor, if present, can be detected, and a diagnosis can be made, by imaging the distribution of labeled stem cells within a subject following administration of the stem cells to the subject. Without being limited by theory, the present inventors propose that BMSCs can be recruited from the circulation into malignant tissues following hypoxic stress or injury, and therefore can serve as probes for such tissues, and in particular the present inventors have observed that human bone marrow mesenchymal stem cells preferentially migrate to ovarian cancer cells.

In various configurations, a cancer which can be detected using compositions and methods of the present teachings can be an ovarian cancer. In various configurations, the BMSCs can be autologous or heterologous to a subject, and the cancer can be an ovarian cancer. BMSCs can be obtained by methods well known to skilled artisans. In particular, autologous stem cells can be obtained by routine methods, and in some configurations, the stem cells can be amplified ex vivo prior to administration to a subject. Furthermore, a subject can be any mammal, such as but not limited to, a human in need of diagnosis or therapy for a cancer; a companion animal such as a cat or dog; an agricultural animal such as a cow, a sheep or a pig, or a laboratory animal such as a mouse or rat.

In other aspects, the present inventors disclose methods and compositions for treating a cancer, such as an ovarian cancer. A cancer of these methods can comprise hypoxic tumors. In various configurations, these methods comprise administering to a subject in need of treatment a therapeutically effective amount of stem cells comprising a therapeutic agent. In various configurations, a therapeutic agent comprised by stem cells can be an enzyme which activates a prodrug, such as, without limitation, the enzyme cytosine deaminase, which can activate the prodrug 5-fluorocytosine (5-FC) to the cytotoxic metabolite, 5-fluorouracil (5-FU). In some configurations, the stem cells can comprise a nucleic acid encoding the therapeutic agent. Without limitation, stem cells of these aspects can comprise a vector comprising a nucleic acid, wherein the nucleic acid comprises a promoter operably linked to a nucleic acid sequence encoding an therapeutic agent such as the enzyme cytosine deaminase. The vector can be any vector known to skilled artisans, such as, without limitation, a virus, a plasmid, or a fragment thereof. Without limitation, a vector can be a plasmid comprising a retroviral pBabePuro backbone (Morgenstern, J. P., Nucl. Acids Res. 18: 3587-3596, 1990; Aboody, K. S., Proc. Nat'l Acad. Sci. USA 97: 12846-12851, 2000). Such vectors and cells can be generated using methods and materials well known to skilled artisans. In various configurations of the disclosed treatment methods, the stem cells can be bone marrow stem cells (BMSCs), which can be autologous or heterologous to the subject. Autologous stem cells can provide several advantages, such as, for example, non-immunogenicity towards the donor/recipient; bone marrow ablation can be avoided; their use may reduce the effective dose of chemotherapeutic drugs; and they may avoid the same ethical issues as embryonic stem cells. Furthermore, in various configurations, a subject can be any mammal, such as but not limited to, a human in need of diagnosis or therapy for a cancer; a companion animal such as a cat or dog; an agricultural animal such as a cow, a sheep or a pig, or a laboratory animal such as a mouse or rat.

In various aspects, a treatment of the present teachings can further comprise administering a therapeutically effective amount of a prodrug to a subject. In addition, some aspects of disclosed methods for treating a cancer such as an ovarian cancer can include obtaining the BMSCs from the subject, and infecting, transfecting, or transforming or the BMSCs with a vector comprising a polynucleotide comprising a promoter operably linked to a sequence encoding an enzyme which activates a prodrug, such as, without limitation, a cytosine deaminase (Danielsen, S., Mol. Microbiol. 6: 1335-1344, 1992). In various configurations, the cytosine deaminase can be a prokaryotic cytosine deaminase, such as an E. coli cytosine deaminase of the following amino acid sequence:

(SEQ ID NO: 1)

msnnalqtii narlpgeegl wqihlqdgki saidaqsgvm pitensldae qglvippfve phihldttqt agqpnwnqsg
tlfegierwa erkallthdd vkqrawqtlk wqiangiqhv rthvdvsdat ltalkamlev kqevapwidl qivafpqegi
lsypngeall eealrlgadv vgaiphfeft reygveslhk tfalaqkydr lidvhcdeid deqsrfvetv aalahhegmg
arvtashtta mhsyngayts rlfrllkmsg infvanplvn ihlqgrfdty pkrrgitrvk emlesginvc fghddvfdpw
yplgtanmlq vlhmglhvcq lmgygqindg lnlithhsar tlnlqdygia agnsanliil paengfdalr rqvpvrysvr
ggkviastqp aqttvyleqp eaidykr.

### EXAMPLES

The following examples provide non-limiting illustrations of the present teachings. While some of examples may include conclusions about the way the invention may function, the inventor do not intend to be bound by those conclusions, but put them forth only as possible explanations. Unless noted by use of past tense, presentation of an example does not imply that an experiment or procedure was, or was not, conducted, or that results were, or were not actually obtained. The experiments utilize standard laboratory techniques and equipment, such as those set forth in Sambrook, J., et al., Molecular Cloning: A Laboratory Manual, 3rd ed. Cold Spring Harbor Laboratory Press, Cold Spring Harbor, N.Y., 2001; Spector, D. L. et al., Cells: A Laboratory Manual, Cold Spring Harbor Laboratory Press, Cold Spring Harbor, N.Y., 1998; and Ausubel, F. M., et al., ed., Current Protocols in Molecular Biology, or as described in a reference cited herein. Except where noted, cell lines are obtained from American Type Culture Collection, Monassas, Va. and maintained according to supplier's instructions.

Many of the experiments presented below use in vitro models, including a) tumor spheroids; b) a chemotaxis migration assay or c) a Matrigel invasion assay. Many of the experiments use human mesenchymal bone marrow stem cells and/or ovarian cancer cell lines Hey, SKOV3, or OCCl.

In some spheroid migration assays, Hey and OCCl tumor cell spheroids are incubated with CFDA-labeled hMSCs or fibroblasts (controls). After 24 h, spheroids are observed under fluorescence illumination. Resultant data indicate that stem cells move toward ovarian cancer cells.

In some chemotaxis migration assays, cloning cylinders are used. In these experiments, cloning cylinder can contain inside labeled human MSCs, while outside a cylinder can contain ovarian cancer cells or fibroblasts (control) or hMSCs (control). A cylinder is removed at 4 hr, and migrated stem cells are counted at 24 hr and 48 hr. Resultant data indicate significant and preferential movement of stem cells toward ovarian cancer cells.

In some invasion assays, Matrigel-coated Boyden chambers are used. A lower chamber contains conditioned medium (CM) such as a cancer cell-conditioned medium, media without serum, or medium+serum. An upper chamber contains a Matrigel-coated membrane and hMSCs. After 48 hr, hMSCs on the upper membrane surface are removed. Migrated cells are fixed, stained and counted. The experiments are run in triplicate. Resultant data indicate that stem cells invade extracellular matrix in response to soluble factor(s) from ovarian cancer cells.

### Example 1

This example illustrates establishment of human bone marrow stem cell (hBMSC) lines and ovarian cancer cell lines.

In these experiments, hBMSC lines from bone marrow aspirates were isolated. Briefly, stem cells were isolated from patient bone marrow aspirates using a ficoll gradient separation technique. Dr. Marc Katchen, Director of the Parkinson's Center, Department of Neurology, Methodist Medical Center, Peoria, Ill. provided the bone marrow aspirates. Dr. Darwin J. Prockop, Tulane Center for Gene Therapy, Tulane University Health Science Center, New Orleans, La., provided bone marrow mesenchymal stem cells. Following centrifugation, stem cells were removed, washed, concentrated and placed into culture at 37° C., in 5% CO₂ and 9% O2. Cells were cultured in human complete culture medium (hCCM) consisting of DMEM (Invitrogen, Carlsbad, Calif.) with 16% fetal bovine serum (FBS, Hyclone, Logan, Utah), L-glutamine (200 mM, Sigma, St. Louis, Mo.), penicillin (100 Units/ml, Invitrogen), and streptomycin (100 μg/ml, Invitrogen). In order to have an adequate stock of cells available for use in these and future experiments, aliquots of hBMSCs (1×10⁶ cells/ml) were cryopreserved in hCCM with 5% DMSO and 30% FBS. Thus far, Ms. Hall and Ms. Steker have banked over 100 vials of these cells.

Two established ovarian cancer cell lines, SKOV3 and HEY, provided by Dr. Mary McAsey, Southern Illinois University School of Medicine, Springfield, Ill. were maintained in minimum essential media (MEM) containing 10% FBS, sodium pyruvate (1 mM/L, Sigma) nonessential amino acids (1× solution, Invitrogen), L-glutamine (200 mM) and a 2-fold vitamin solution (Invitrogen, Carlsbad, Calif.). The cells were maintained at 37° C. in 5% CO₂. In order to have an adequate stock of cells available for use in experiments, aliquots of these cell lines were cryopreserved in MEM with 5% DMSO and 10% FBS. Thus far, we have banked over 100 vials of each cell line for future experiments.

### Example 2

This example illustrates that human bone marrow stem cells migrate toward soluble factors in culture media.

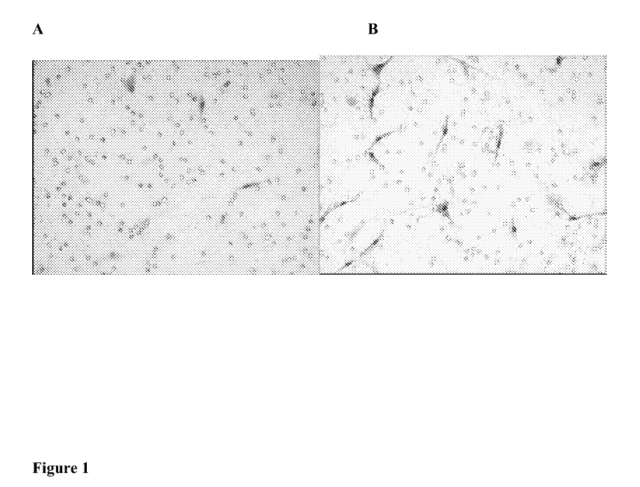

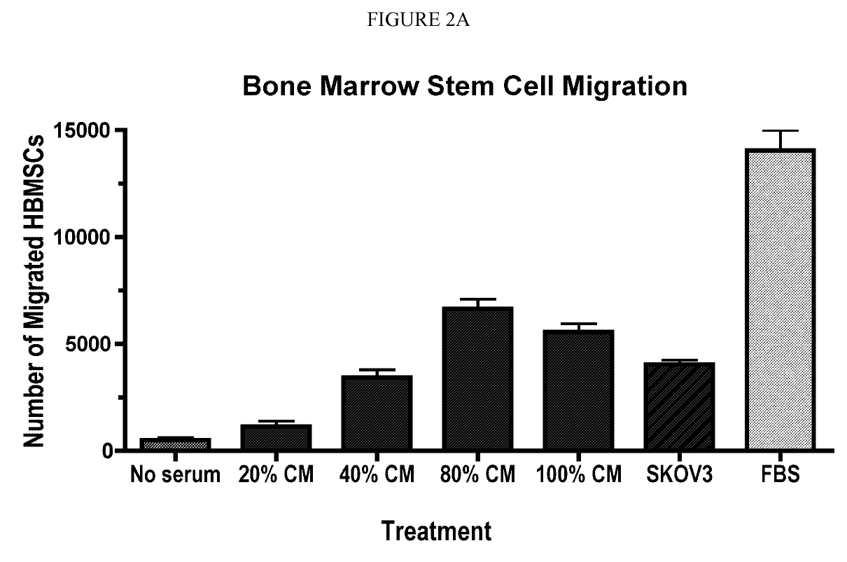

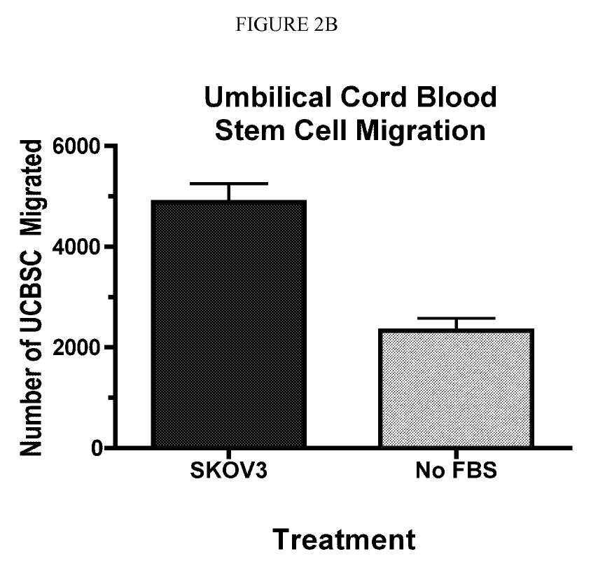

To assess the chemotaxis response of stem cells in vitro, we used a modified Boyden chamber that consisted of upper and lower wells separated by a small matrigel-coated filter membrane with 8 μm pores. Human BMSCs were cultured to 50% confluence and harvested using trypsin. Cells were washed with sterile phosphate buffered saline, and 200 μl of hBMSCs (1×10³) were diluted in hCCM with 16% FBS and added to the upper compartment of a Biocoat matrigel invasion chamber (BD Biosciences, San Jose, Calif.). A total of 750 μl of hCCM with or without 16% FBS was added to the lower chamber and the cells were incubated at 37° C., 5% CO₂, 9% O₂ for 48 hours. After incubation, the cells on the upper surface of the membrane were removed with a cotton tip applicator. Cells that had migrated through the membrane were fixed in 4% para-formaldehyde for 3 minutes, washed twice in PBS, stained with crystal violet and the stained cells were observed using an inverted microscope. The number of migrating cells was assessed by counting cells in a minimum of 3 random fields (20× magnification) per membrane. Stem cells cultured in media without 16% FBS in the lower chamber exhibited little migration (5.6±1.5 cells/field), whereas stem cells cultured in media with 16% FBS exhibited robust migration (93.3±13.1 cells/field, p<0.001) These two treatment groups serve as positive and negative controls to examine the effect of soluble factors from ovarian cancer cell lines on the tropic response of hBMSCs. Illustration of Human HBMSC migration through 8 μm pores in a Matrigel assay is presented in FIG. 1. Quantified results of bone marrow stem cell migration in response to conditioned media from ovarian cancer cells is presented in FIG. 2.

### Example 3

This example illustrates that human bone marrow stem cells migrate to ovarian cancer multicellular tumor spheroids in vitro.

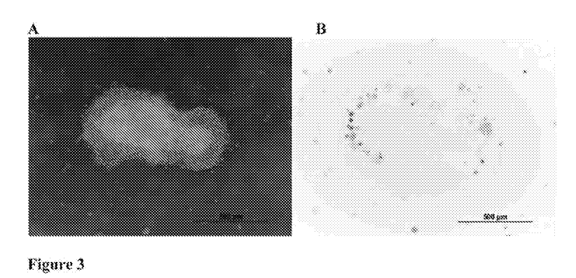

Compared to monolayer cultures, multicellular tumor spheroids (MCTS) may better model solid tumors. MCTS mimic the early small, avascular micrometastases of ovarian cancer observed in vivo. MCTS consist of three cell populations similar to that of microregions of solid tumors. These include cells that are actively dividing at the periphery, an intermediate population of quiescent, yet viable cells and a central, necrotic region (Kunz-Schughart and Muller-Klieser, 2000). Because of this, we examined whether hBMSCs preferentially migrate toward tumor spheroids. For generation of spheroids, Hey ovarian cancer cells (1×10⁵ cells) were plated on 100 mm agarose-coated Petri dishes and cultured at 37° C., 5% CO₂, 9% O₂ for four days, avoiding any motion of dishes. On day three of culture, hBMSCs were labeled with 5-(6)-carboxyfluorescine diacetate, succinimidyl ester (CFDA-SE, Invitrogen) and 1×10⁴ cells were plated into several wells of a 24-well culture dish. CFDA-SE is a cell permeant molecule that is converted by intracellular esterases to anionic CFSE. Intracellular proteins stably bind to CFSE for more than 8 cell divisions. Other wells received the same concentration of CFDA-SE-labeled non-tumorigenic Detroit-551 lung fibroblast cells. On day four, three to five spheroids, 300-500 um in diameter were then added into each well of the 24-well culture dish. After 24 hours spheroids were observed under fluorescence illumination and assessed for stem cell or fibroblast cell attachment. Stem cells migrated toward and attached to the spheroids at a much higher concentration than fibroblast cells. Illustration of migration of human bone marrow stem cells toward Hey spheroids is presented in FIG. 3.

### Example 4

This example illustrates in vitro tropism of stem cells in co-cultures of ovarian cancer cells. In these experiments, a sterile glass cylinder, 60 mm outer diameter, (Fisher Scientific, Pittsburgh, Pa.) is attached using sterile silicone grease to each poly-L-lysine-coated glass coverslip. Hey or SKOV3ip.1 cells (1×10⁵ cells) are plated onto these coverslips. Human BMSCs (1×10⁴ cells) are labeled with CFDA-SE then seeded into the central area of the cylinder and allowed to adhere to the coverslip. For control experiments, Detroit 551 fibroblast cells are plated in a similar manner as Hey or SKOV3ip.1 cells. On other coverslips, a second cylinder is placed over the adherent Hey, SKOV3ip.1 or fibroblast cells and a similar number of stem cells is plated directly on top of the monolayer of these cells. The cylinders are removed on the day after plating of stem cells and the coverslips are cultured for an additional 1-3 days. On days 1-3 following plating of stem cells, the cells are examined using a fluorescence microscope (Olympus, Inc., Center Valley, Pa.) and the migration distance of stem cells, from the center of the cylinder to the outermost periphery in which stem cells were observed are measured using Microsuite/DP70 image analysis software (Olympus).

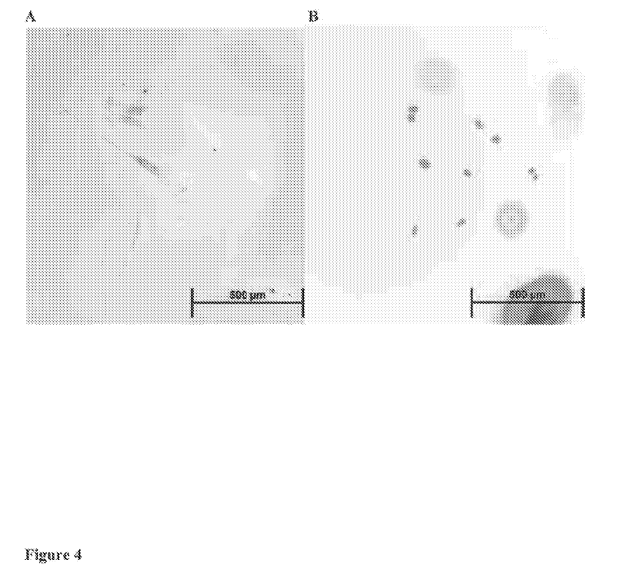

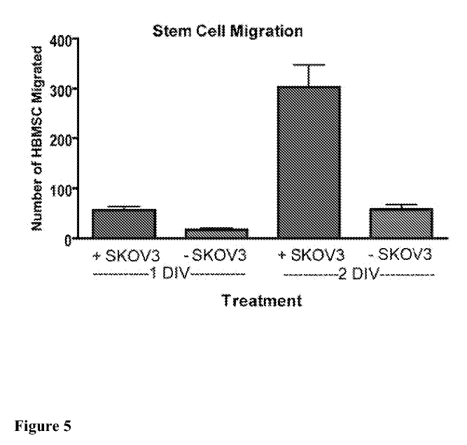

FIG. 4 illustrates an example of coculturing HBMSC and SKOV3 cells.

### Example 5

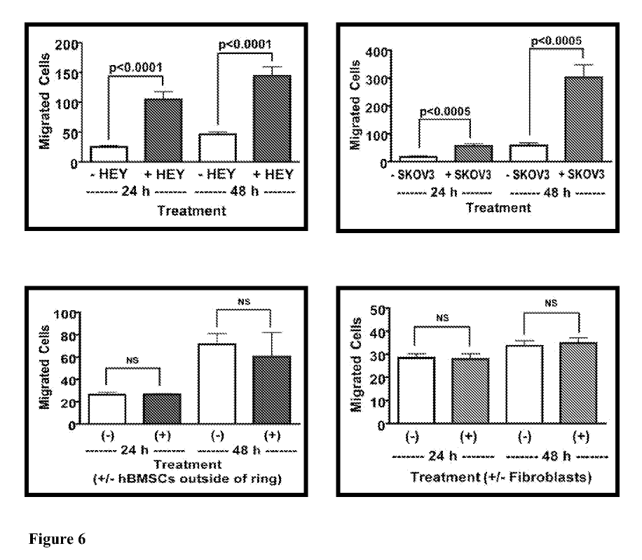

This example illustrates in vitro stem cell migration in response to soluble chemotactic factors secreted by ovarian tumor cell lines. In these experiments, Hey or SKOV3ip.1 cells at 70% confluence are incubated in serum-free medium for 24-48 h. This conditioned medium is then aspirated, diluted and placed into the lower wells of the invasion chambers. Other aliquots of conditioned media are heat inactivated, diluted and placed into the lower wells of the invasion chambers. hMSCs in serum-free medium are placed in the upper chambers of a 24-well invasion chamber. Positive and negative controls consist of media with and without 16% FBS, respectively in the lower wells of the invasion chambers. Stem cells are cultured for 24-48 h in 5% CO₂ 9% O₂ at 37° C. In other experiments, Hey or SKOV3ip.1 cells are cultured in the bottom wells of the invasion chambers, serum-starved for 48 h after which hMSCs in serum-free medium are placed in the upper chambers and cultured for an additional 24-48 h. FIG. 6 illustrates enhanced migration of human bone marrow stem cells when co-cultured with HEY cells.

### Example 6

This example illustrates results of some chemotaxis migration assays using cloning cylinders as outlined above. The data, as shown in FIG. 6 indicate a strong positive influence on cell migration by ovarian cancer cells Hey or SKOV3, but not by control fibroblasts.

### Example 7

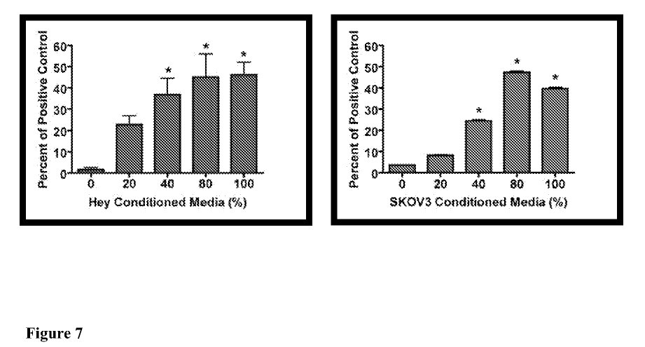

This example illustrates results of some Matrigel invasion assays described above. The data (FIG. 7) show that conditioned media from Hey or SKOV3 ovarian cancer cells provide component(s) which stimulate matrigel invasion.

### REFERENCES

The following references may provide additional background information and methodologies used herein.
Khakoo A Y, Pati S, Anderson S A, Reid W Elshal M F, Rovira I I, Nguyen A T et al. Human mesenchymal stem cells exert potent antitumorigenic effects in a model of Kaposi's sarcoma. J Exp Med. 2006; 203: 1235-47.
Kim S K, Cargioli T G, Machluf M, Yang W, sun Y, Al-Hashem R, Kim S U, Back P M, Carroll R S. PEX-producing human neural stem cells inhibit tumor growth in a mouse glioma model. Clin Cancer Res. 2005; 11: 5965-70.
Kunz-Schughart L A, Mueller-Klieser. Three-dimensional culture. In: Animal Cell Culture, 3^{rd} edition. (ed. JRW Masters), p. 315. Oxford University Press, 2000.
Nakamizo A, Marini F, Amano T, Khan A, Studeny M, Gumin J, Chen J, Hentschel S, Vecil G, Dembinski J, Andreeff M, Lang F F. Human bone marrow-derived mesenchymal stem cells in the treatment of gliomas. Cancer Res. 2005; 65: 3307-18.
Schmidt N O, Przylecki W, Yang W, Ziu M, Teng Y, Kim S U, Black P M, Aboody K S, Carroll R S. Brain tumor tropism of transplanted human neural stem cells is induced by vascular endothelial growth factor. Neoplasia. 2005; 7: 623-9.
Schichor C, Birnbaum T, Etminan N, Schnell O, Grau S, Meibach S, Aboody K, Padovan C, Straube A, Tonn J, Goldbrunner R. Vascular endothelial growth factor A contributes to glioma-induced migration of human marrow stromal cells (hMSC). Exp Neuro. 2006; 199: 301-10.
Tabatabai G, Frank B, Mohle R, Weller M, Wick W. Irradiation and hypoxia promote homing of haematopoietic progenitor cells towards gliomas by TGF-beta-dependent HIF-1 alpha-mediated induction of CXCL12. Brain. 2006; 129: 2426-35.

All publications and patent applications cited in this specification are herein incorporated by reference as if each individual publication or patent application were specifically and individually indicated to be incorporated by reference. Although the foregoing teachings have been described in some detail by way of illustration and example for purposes of clarity of understanding, it will be readily apparent to those of ordinary skill in the art in light of the teachings that certain changes and modifications may be made thereto without departing from the spirit or scope of the appended claims.

What is claimed is:

1. A method of detecting a cancer, comprising:
administering a plurality of labeled stem cells to a subject having, or suspected of having, a cancer; and
detecting the distribution of the stem cells.

2. A method of detecting a cancer in accordance with claim 1, wherein the labeled stem cells comprise nanoparticles.

3. A method of detecting a cancer in accordance with claim 2, wherein the nanoparticles are mono-crystalline iron oxide nanoparticles (MIONS).

4. A method of detecting a cancer in accordance with claim 3, wherein the detecting the distribution of the stem cells comprises imaging the MIONS.

5. A method of detecting a cancer in accordance with claim 4, wherein the imaging comprises magnetic resonance imaging (MRI).

6. A method of detecting a cancer in accordance with claim 1, wherein the stem cells are bone marrow stem cells (BMSCs).

7. A method of detecting a cancer in accordance with claim 6, wherein the BMSCs are autologous to the subject.

8. A method of detecting a cancer in accordance with claim 1, wherein the cancer is an ovarian cancer.

9. A method of detecting a cancer in accordance with claim 1, wherein the subject is a mammal.

10. A method of detecting a cancer in accordance with claim 9, wherein the mammal is a human.

11. A method for treating an ovarian cancer, comprising administering to a subject in need of treatment a therapeutically effective amount of stem cells comprising a therapeutic agent.

12. A method of treating an ovarian cancer in accordance with claim 11, wherein the therapeutic agent is an enzyme which activates a prodrug.

13. A method of treating an ovarian cancer in accordance with claim 12, wherein the prodrug is 5-fluorocytosine and wherein the enzyme which activates the prodrug is a cytosine deaminase.

14. A method of treating an ovarian cancer in accordance with claim 11, wherein the stem cells are bone marrow stem cells (BMSCs).

15. A method of treating an ovarian cancer in accordance with claim 14, wherein the BMSCs are autologous to the subject.

16. A method of treating an ovarian cancer in accordance with claim 11, wherein the subject is a mammal.

17. A method of treating an ovarian cancer in accordance with claim 16, wherein the mammal is a human.

18. A method of treating an ovarian cancer in accordance with claim 12 or claim 13, further comprising administering a therapeutically effective amount of the prodrug to the subject.

19. A method of treating an ovarian cancer in accordance with claim 15, further comprising:
obtaining the BMSCs from the subject; and
infecting, transfecting, or transforming or the BMSCs with a vector comprising a polynucleotide comprising a promoter operably linked to a sequence encoding an enzyme which activates a prodrug.

20. A method of treating an ovarian cancer in accordance with claim 19, wherein the enzyme is a cytosine deaminase.

21. Bone marrow stem cells comprising mono-crystalline iron oxide nanoparticles.

22. Bone marrow stem cells in accordance with claim 21, wherein the stem cells are autologous to an intended recipient.


SEQUENCE LISTING (1 sequence)

SEQ ID NO: 1 - protein, 427 aa, Escherichia coli
MSNNALQTIINARLPGEEGLWQIHLQDGKISAIDAQSGVMPITENSLDAEQGLVIPPFVEPHIHLDTTQTAGQPNWNQSGTLFEGIERWAERKALLTHDDVKQRAWQTLKWQIANGIQHVRTHVDVSDATLTALKAMLEVKQEVAPWIDLQIVAFPQEGILSYPNGEALLEEALRLGADVVGAIPHFEFTREYGVESLHKTFALAQKYDRLIDVHCDEIDDEQSRFVETVAALAHHEGMGARVTASHTTAMHSYNGAYTSRLFRLLKMSGINFVANPLVNIHLQGRFDTYPKRRGITRVKEMLESGINVCFGHDDVFDPWYPLGTANMLQVLHMGLHVCQLMGYGQINDGLNLITHHSARTLNLQDYGIAAGNSANLIILPAENGFDALRRQVPVRYSVRGGKVIASTQPAQTTVYLEQPEAIDYKR